(19) United States
(12) Patent Application Publication
(10) Pub. No.: US 2023/0252510 A1
(43) Pub. Date: Aug. 10, 2023

(54) SYSTEMS AND METHODS FOR BLOCKCHAIN-BASED INFORMATION SHARING

(71) Applicant: UMedHub, Inc., Gulf Breeze, FL (US)

(72) Inventor: Sergey Kachur, Gulf Breeze, FL (US)

(21) Appl. No.: 18/106,162

(22) Filed: Feb. 6, 2023

Related U.S. Application Data
(60) Provisional application No. 63/306,865, filed on Feb. 4, 2022.
(60) Provisional application No. 63/435,114, filed on Dec. 23, 2022.

(51) Int. Cl. G06Q 30/0208; G06Q 50/00; G06Q 20/38; G06Q 20/12

(52) U.S. Cl. CPC G06Q 30/0208; G06Q 50/01; G06Q 20/389; G06Q 20/1235

(57) ABSTRACT

Systems and methods are disclosed for managing information. In certain embodiments, a server receives one or more information queries and associated entries and stores references to them as well as associated metadata as a smart contract. The server then modified references and metadata of the entries/queries based on expert and user interactions. The economic activity of these interactions is guided by organizations of self-governing expert participants who are responsible for setting prices, community policies, and credentialling requirements. Over-time the incentive mechanisms of the protocol, incentive mechanisms associated with governance and constant improvement of input/output algorithms via neural network analyses of iterative user-system interactions optimize the function and increase the value of the knowledge economy, creating vibrant knowledge marketplaces that collect fees in exchange for data distribution to users and distribute those fees among expert individuals and their knowledge communities.

### CROSS-REFERENCE TO RELATED APPLICATIONS

This application claims the benefit of U.S. Provisional App. No. 63/306,865, filed Feb. 4, 2022, and U.S. Provisional App. No. 63/435,114, filed Dec. 23, 2022, the entire contents of which are incorporated herein by reference.

### FIELD OF THE INVENTION

The present invention is directed to the field of a blockchain-based information sharing and economy system designed around economic incentives for professional database curation to optimize information access for society.

### BACKGROUND OF THE INVENTION

Today's society is characterized by hyperconnectivity. Through our phones and computers, we are connected 24/7 to 4.3 billion people and 94 trillion gigabytes of information. Needless to say, not all of this information can be effectively converted into useful knowledge, especially since many millions of these gigabytes contain disinformation. Public health and safety are more costly and jeopardized daily by the spread of this disinformation. In a worst-case scenario, this can even cost lives. Re-imagine the false vaccination studies that made the rounds since 2000 or the fake information about the Russia/Ukraine war.

Solutions to the current information crisis are insufficient. Media companies and social networks claim to help users find their way via curated content. Yet, their business models are heavily optimized for generating user engagement and upselling additional services. A study by Global Disinformation Index (GDI) found that in 2020, companies like Google & Amazon paid out ad revenues of about $25 million to websites that produce false or misleading information. Finding the best qualitative answer to a question on the internet remains an empty marketing claim.

Blockchain systems are an emerging mechanism of information security designed for global access. Current uses include numerical information storage, currency applications (cryptocurrency), computing cooperation algorithms, complex debt instruments, personal data security, digital asset storage, automated market analysis and optimization algorithms, and others. Yet, no decentralized and expertly curated information delivery systems have been developed.

To-date, there are no good solutions to capture and steer the value of producing high-quality information beyond expensive subscription-based business models or paid 1-on-1 consultations with experts. The presence of intermediaries creates economic inefficiencies as they extract an additional share from the business, not infrequently at inflated prices. Examples include the New England Journal of Medicine and other subscription-based scientific journals. Perhaps the experiment that most attempted to create quality information in a decentralized way is Wikipedia. In many ways, this effort has been successful, but the platform still depends on donations for external revenue sources and volunteers who have no extrinsic incentive for quality control. The most controversial issues are constantly being rewritten.

Blockchain technology is most widely known as the technology behind the popular cryptocurrencies, such as Ethereum and Bitcoin. A blockchain creates a history of data deposits, messages, or transactions in a series of blocks where each block contains a hash of the previous block. That creates a chain where any changes made to a block will change that block's hash, which must be recomputed and stored in the next block. That changes the hash of the next block, which must also be recomputed throughout until the end of the chain.

Although the hash is simple to compute, there are rules imposed which require the value of the hash to be below a certain threshold value. In addition, the hash is based on a special type of mathematical function that is not reversible, such that only brute force calculations must be used to “solve” the hash. A valid hash is found by repeatedly adjusting a changeable value in the block and recalculating the hash until it meets the validity requirements. The freely changeable value may be referred to as a nonce. The unpredictable nature of the hash considerably increases the difficulty of finding a nonce that produces a valid hash of the block. Typically, trillions of different nonces must be tried before a valid hash is found.

The security of a blockchain is further increased by implementing it on a distributed network. That means that many users all have access to the blockchain and are all attempting to add blocks to the end of the chain by finding a nonce that produces a valid hash for a given block of data. When two blocks are found that both claim to reference the same previous block, a fork in the chain is created. Some users in the network will attempt to find the next block on one end of the fork while other users will work from the other end of the fork. Eventually, one of the forks will surpass the other in length, and the longest chain is accepted by consensus as the valid chain. Each new block contains a reference to the prior block, a counter, and transaction data generated on the network. Therefore, anyone who attempts to change a block must not only re-find a valid hash for each subsequent block but must do it faster than everyone else working on the currently accepted chain. Thus, after a certain number of blocks have been chained onto a particular block, it becomes prohibitively costly to try to change that block.

Global information access is significantly less novel since the development of global network communication protocols and the rise of the commercial internet. However, information security and information authenticity has been one of the limitations associated with this system. The current global financial landscape incentivizes centralized data aggregation and user engagement rather than data curation and quality. The result is an unfiltered deluge of information available with low levels of curation and auditing that yields a near limitless number of narratives and dilutes the value and reduces acceptance of consensus findings.

Financial rewards provide a solution to this issue. Financial rewards incentivize the securing of information within each block. The cryptographic key for each block is generated via solving SHA-256 hashes as a result of either computing power (proof-of-work), or validated stakes locked in-network (proof-of-stake). That contribution to security is rewarded by the distribution of rewards (newly generated tokens or coins) and with fees paid by network users.

Blockchains on a distributed network with sufficiently restrictive rules for creating valid blocks are extremely secure against unauthorized changes to the data stored in them. That makes blockchains particularly useful for recording sensitive information.

One such example is the field of health information, where fidelity and accuracy of is crucial to patient treatment and the management of resources. Therefore, there is a need for a system that uniquely incorporates blockchain to address these and other deficiencies.

### SUMMARY OF THE INVENTION

The present invention comprises techniques for managing information. In certain embodiments, a server receives one or more information queries and stores their contents or references to them in a smart contract. The server then receives one or more responses to the information queries that it references in the smart contract. The smart contract interacts with other smart contract modules that manage economy, governance, and data access information and are recomputed to update the state and associated values with each block. This information is computed by front-end applications/servers using a neural network algorithm as an iterative learning tool to optimize system responses to user requests for information with the aim of generating iteratively better (faster and more relevant) outputs.

In an economic sense, this invention provides a framework in which experts and initial question askers generally are expecting future rewards from their contributions while users solely accessing answers to questions are paying for a service provided by initial question askers and experts.

In certain embodiments, the user posts an original query not found in the information network, and the creation of a new query within the smart contract requires a staked fee.

In other embodiments, the user transmits a question that matches an existing query in the network with a fee and that fee is divided among the original author of the query, the slashing pool, and a positively flagged (endorsed) expert entry in response to that query.

In certain embodiments, the verified expert contributors pay a fee in order to add an entry that has their expert opinion to a specific query.

In other embodiments, the verified expert contributors receive compensation for posting entries, to a query.

In yet other embodiments, the blockchain network utilizes proof-of-stake protocols for validation requests.

In certain embodiments, the verified expert contributors receive compensation for flagging misinformation via an equal division of bounty funds from an entry's slashing pool.

In other embodiments, verified information that remains unflagged accrues value and in reaching a pre-defined cap, excess accrual of value within the slashing bounty is transferred to the protocol management organization (DAO).

In certain embodiments, the invention comprises a unique self-curating network of expert consensus rooted information secured by decentralized financial incentives and payments enabled by blockchain technology.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a diagram of an exemplary embodiment of the hardware of the system of the present invention;

FIG. 2A is a diagram showing exemplary interactions within the decentralized database with respect to the roles within the system;

FIG. 2B is a diagram showing aditional exemplary interactions within the decentralized database with respect to the roles within the system;

FIG. 2C is a diagram showing additional exemplary interactions within the decentralized database with respect to the roles within the system;

FIG. 3 is an overview diagram showing the relationships between queries and entries within the knowledge base;

FIG. 4 is a diagram outlining some of the key components of metadata stored in the knowledge base;

FIG. 5A is an exemplary diagram of incentive mechanisms of the system of the present invention;

FIG. 5B is an exemplary diagram of the protocol-based management of the incentive mechanisms through the interaction of smart contract modules;

FIG. 6 is an exemplary economic value flow facilitated by components/modules of the protocol;

FIG. 7 is a diagram summarizing the top-level economic, governance, and organizational structure of the knowledge economy;

FIG. 8 is a diagram summarizing subDAO economic, governance, and organizational structure;

FIG. 9 is a diagram depicting high-level economic interactions between organizations embedded in the knowledge protocol; and

FIG. 10 is a diagram showing the governance architecture of an exemplary embodiment of the present invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

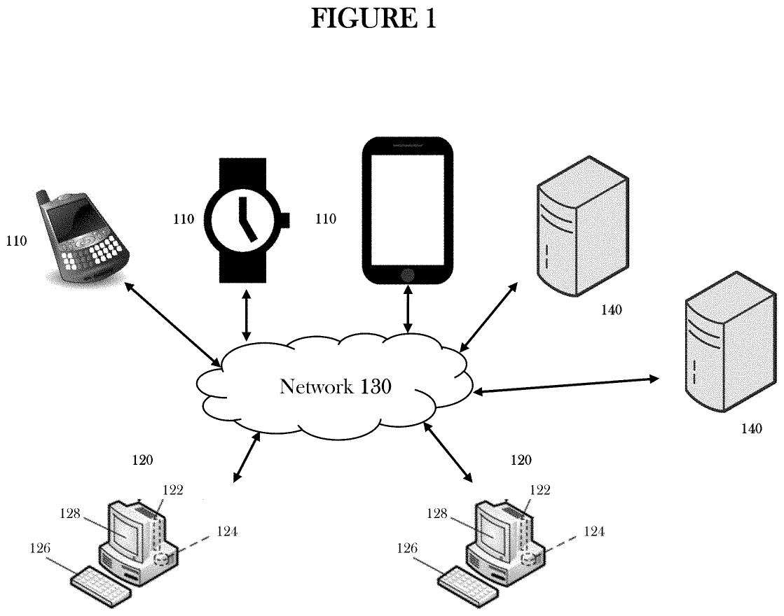

FIG. 1 is an exemplary embodiment of the information system of the present invention. In the exemplary system 100, one or more peripheral devices 110 are connected to one or more computers 120 through a network 130. Examples of peripheral devices/locations 110 include smartphones, tablets, wearables devices, and any other electronic devices that collect and transmit data over a network that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices/locations 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 110 and each of the computers 120 of the system may have software related to the system installed on it. In such an embodiment, system data may be stored locally on the networked computers 120 or alternately, on one or more remote servers 140 that are accessible to any of the peripheral devices 110 or the networked computers 120 through a network 130. In alternate embodiments, the software runs as an application on the peripheral devices 110.

TABLE I

| | |
|---|---|
| User | Main role is to ask relevant questions |
| | Pays a token fee to post a question |
| | (search query) |
| | Pays a token fee to access entries to an existing |
| | question |
| | Receive rewards from access fees whenever future |
| | users access their posted questions |
| Experts/Curators | Answer relevant questions to build the knowledge |
| | base and as slash invalid entries as a means of curation |
| | Can entry questions for small token stakes |
| | Receive rewards from access fees whenever future |
| | users access their answers |
| | (proportional to endorsements) |
| | Able to access answers of the subDAO at reduced |
| | or waived access fees. |
| | Can vote to slash existing answers using token stakes |
| | Get rewarded token fees from a slashing event |
| | Permissions for interacting with entries regulated |
| | by subDAO |
| Knowledge | Group of pre-defined affiliates (e.g. employees) |
| Community | which main role is to onboard users & experts |
| organizers | Access user & expert list in order to send them |
| | tokens to get started |
| | Access to tools for group onboarding of experts |
| | (e.g. by mass verification) |
| | Account with expert/curator privileges |

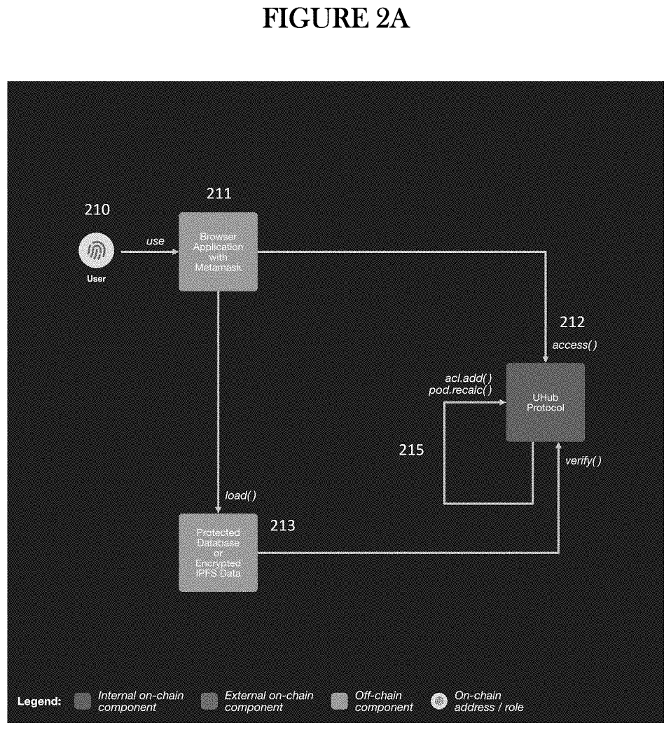

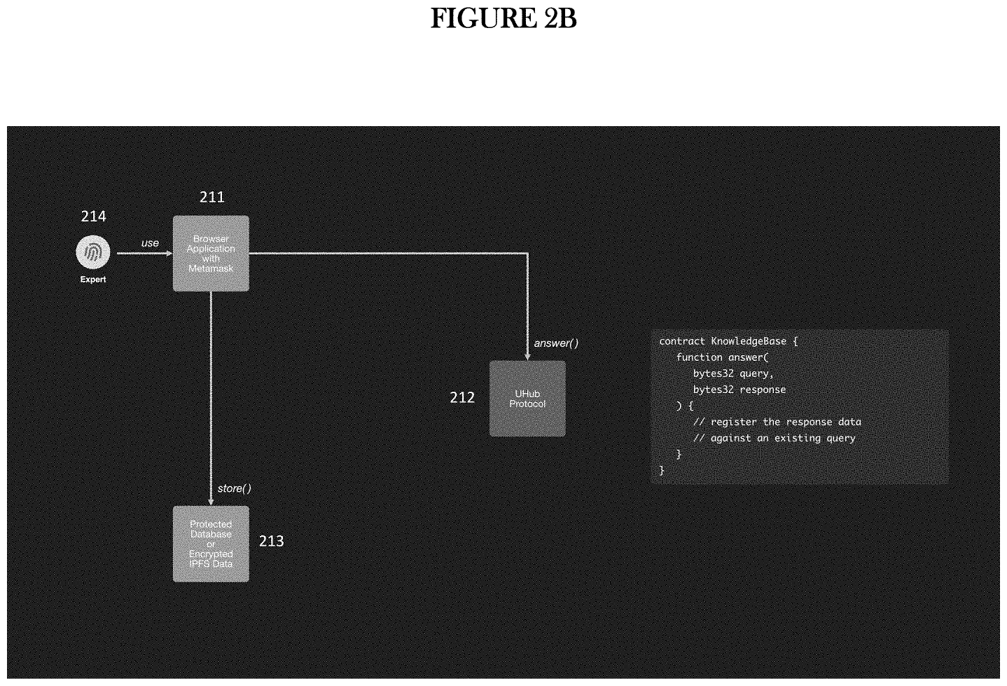

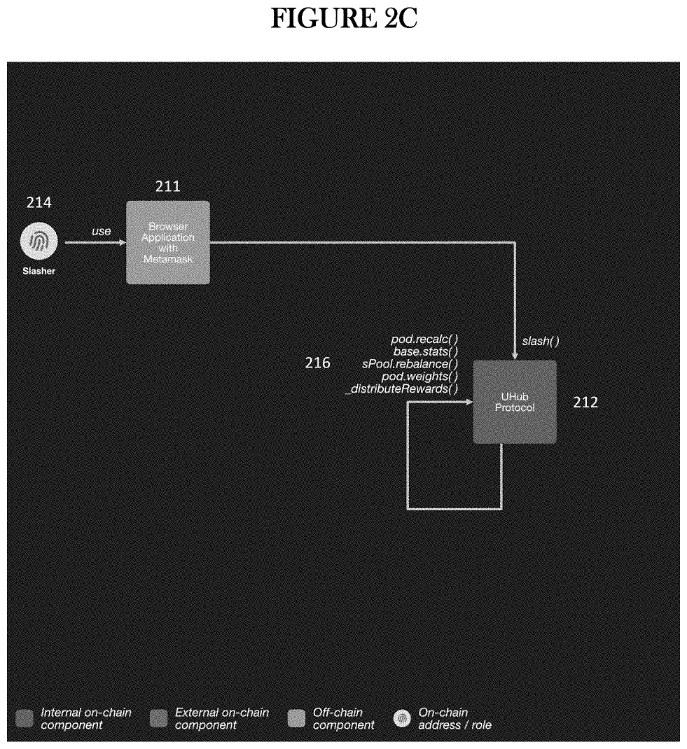

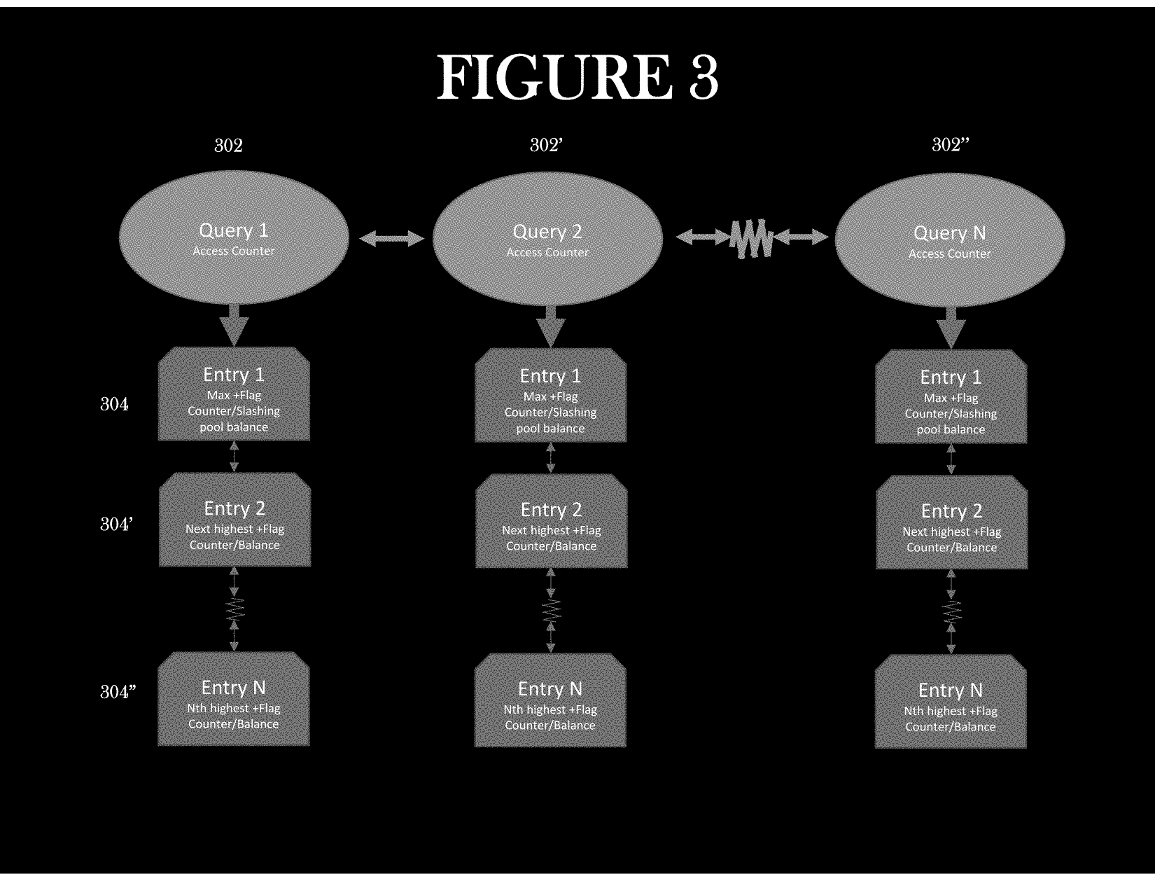

FIGS. 2A through 2C are exemplary diagrams showing the interactions of users and contributors within the information system. In one embodiment, as shown in FIG. 2A, any user wallet address 210 represents user requests for information from their device to be analyzed for relevance by a neural network algorithm via an application 211 that parses data from the information system 212 (named in this instance, UHub protocol) through an application capable of interacting with smart contracts 211 (such as a Metamask browser plugin). This application 211 parses protocol data to return the most relevant data sources 213 based on the neural network algorithm analysis (described with regard to FIG. 3 below) of the most relevant and highly ranked data from the information system 212. The interaction of the application 211 with the information system 212 triggers a recalculation of protocol parameters associated with the specific entry in a query array (or other data structure) 215 that is associated with the data source 213, and all states are updated in the information system during the subsequent block. In another embodiment, any user 210 can request to create a new query within the information network 212 and attach a bounty to attract expert responses in the form of entries. In such an embodiment any user can write a novel query by sending an economic bounty attached to their novel query via their application 211 (permanently associating that query with user's crypto address) in the information network 212. In another embodiment, as shown in FIGS. 2B and 2C, a user wallet address that represents a verified expert contributor 214 is allowed a separate set of interactions with the information system 212. In one embodiment the verified expert contributor can write an entry in response to a query and associate it with an external data source 213 by attaching an economic stake to their entry. This interaction of the application 211 with the information system 212 permanently adds that entry (associated with verified expert contributor's crypto address) to a specific query array (or other data structure), and all states are updated in the information system during the subsequent block. In another embodiment, the verified expert contributor triggers an interaction with an entry in the information system 212 wherein a personal economic stake is added to the balance in the slashing pool 216 of that entry. This interaction of the verified expert contributor's application 211 with the information system 212 triggers a recalculation of the system parameters associated with the specific entry 216 that is associated with its corresponding data source 213, and all states are updated in the information system during the subsequent block. If the number of experts/slashers 214 voting to slash that entry reaches a predefined threshold, the balance of that slashing pool is equally distributed to all stakeholders that voted to slash that specific entry 214.

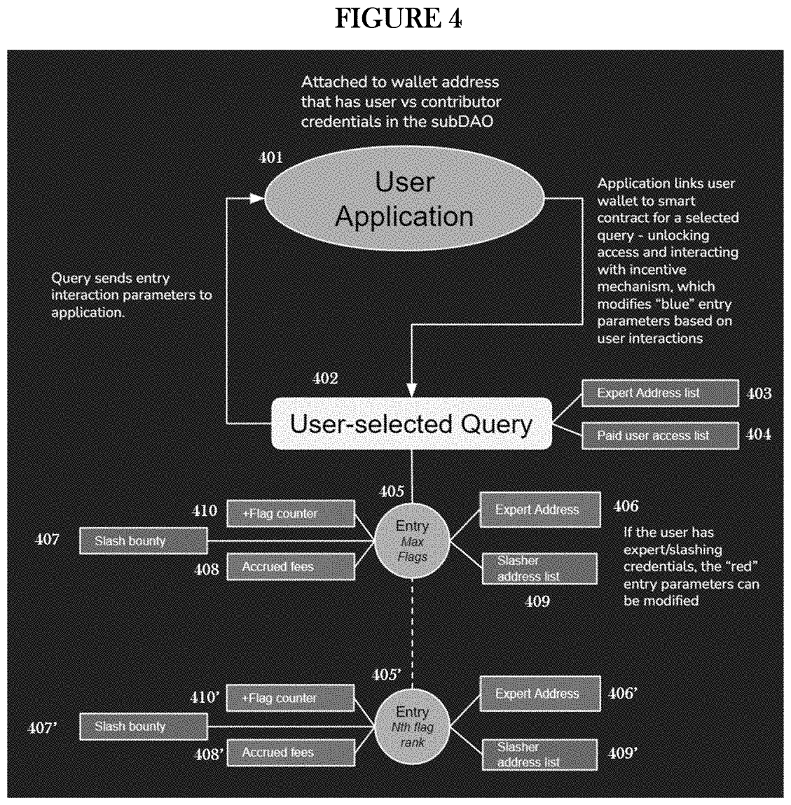

FIG. 3 is an overview flow diagram showing the relationships between queries and entries in an exemplary embodiment of the invention. Questions 302, 302′, 302″ enter the system and receive responses as entries 304, 304′, 304″. Queries are ranked by the number of user interactions; in some embodiments these can be recorded as either a counter of the number of times the query has been accessed or in other embodiments, the amount of economic activity that query has generated (represented as the sum paid to the originator of that query). The answers are ranked exemplarily based on the number of positive flares they receive, which in some embodiments can be the number of positive flags 304 and in other embodiments by the balance in the slashing pool 518 of each entry. When a query 302 is displayed, it is done so based on parsing through a neural network algorithm that analyzes a query's popularity (using blockchain metadata in FIG. 3 and FIG. 4) with respect to relevance to the user's interest. The algorithm collects relevant variables on user behavior from interactions with the front-end application. These variables include but are not limited to number of searches (iterations) of a user-question prior to purchasing access to a relevant existing query in the database, the ranking of the most popular query based on its access counter within a matrix of syntax matches to other queries within the knowledge base to generate a priority list of the most relevant queries to the user's input, number of entries visited prior to positive flagging, etc. With optimal endpoints that include but are not limited to: reducing time spent on searching, improving relevance of search results, reducing time to positively flag an entry within a query, thus streamlining using experience and promoting engagement with the information content as opposed to navigation of the knowledge base. In certain embodiments, support vector machines (SVM), Naïve Bayes classifiers, or decision trees may be used instead of or in addition to the neural network.

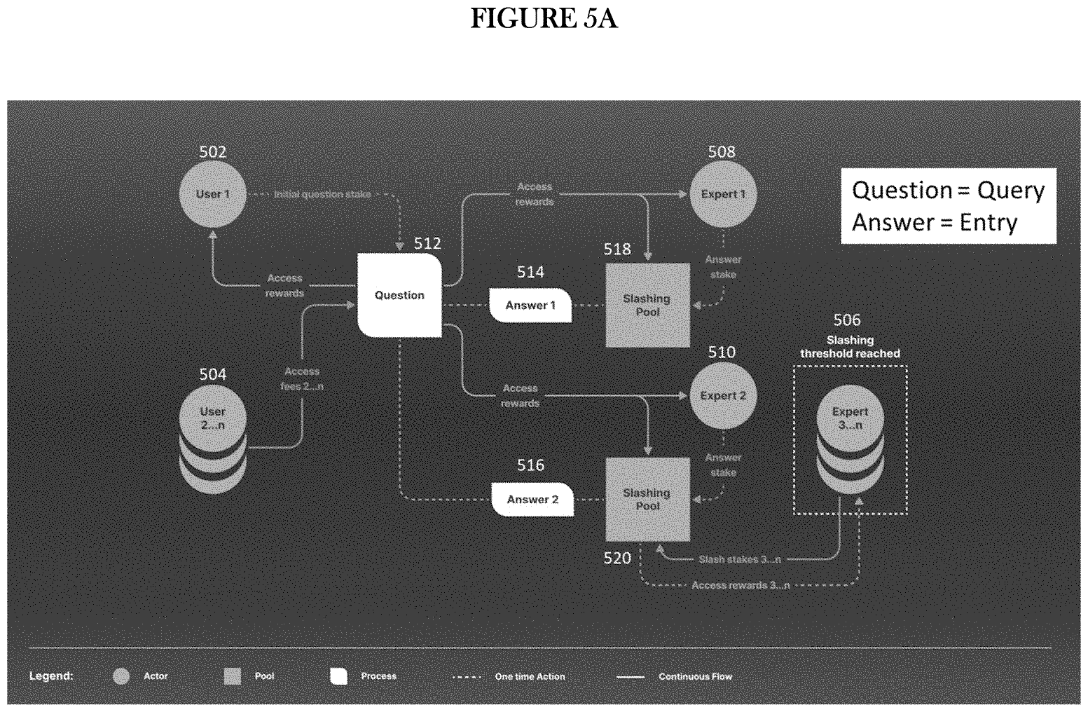

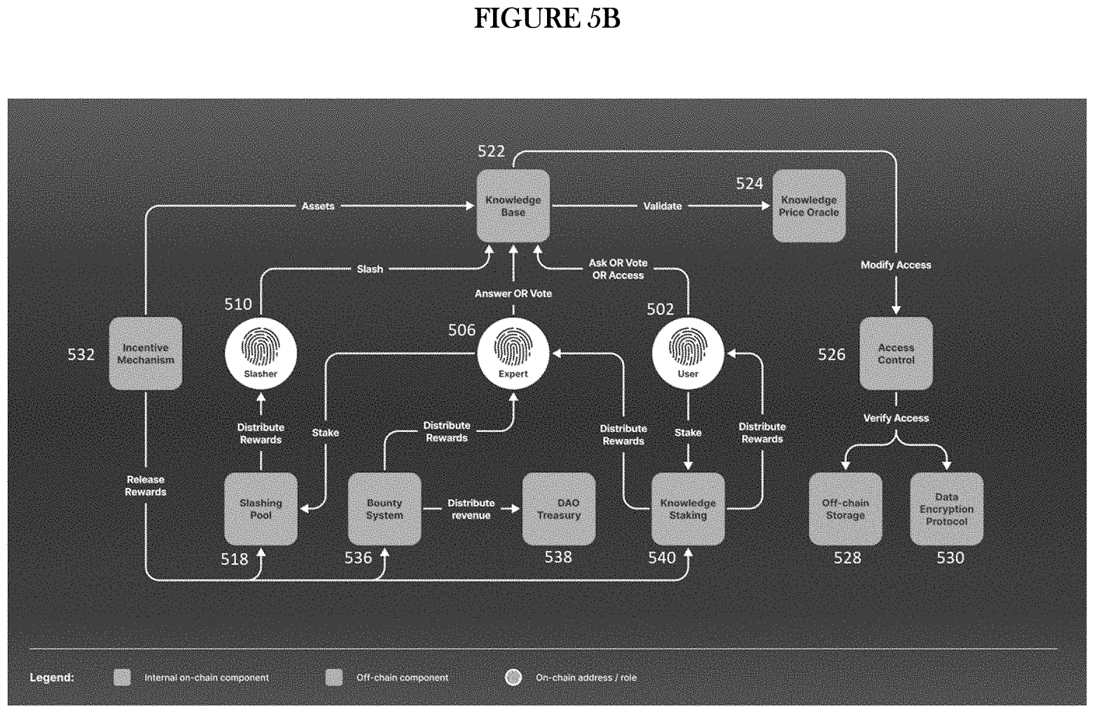

FIG. 4 details the components of some of the structures within the knowledge base of the information system. As specified in FIG. 2, the user interacts with the information system via an application 401 that has permission to interact with the user's cryptographic identity/wallet (described with regard to FIGS. 2A through 2C above) and can parse the user query with respect to on-chain data from the protocol using a neural network algorithm described with regard to FIG. 3 above. Once a user chooses a query 402 with which to interact, the interaction is governed based on permissions associated with address lists 403 and access lists 404 attached to that query 402 that are processed by the access module (described in further detail in FIG. 5B with regard to the access module 526). This action engages the incentive module (described in further detail in FIG. 5B with regard to the incentive module 532, and paragraph 053), splitting the paid fee into portions divided between the query and its author address 402, as well as an entry 405, 405′ and associated expert contributor addresses 406, 406′, and the slashing bounty 407, 407′. The contents of an entry 405, 405′ consist of instructions for accessing any external piece of data and can include but are not limited to: IPFS pointer, a web address, a hosted video/audio/picture file, instructions for accessing private information that can include encryption/decryption keys. If the interacting user has expert address credentials 406, 406′, via the access module 526, they may have access to any public queries agreed upon within the jurisdiction of their subDAO (a decentralized autonomous organization responsible for maintaining access lists to queries pertaining it its specific field of expertise). An expert user, through permissions granted via the access module, can also write entries, and slash or deprioritize an entry 405, 405′, this fee 408, 408′ is added to the slashing bounty 407, 407′, and their address is added to a list of addresses of others that chose to pay this fee 410, 410′. Should the slashing address list reach a predetermined threshold, the slashing bounty 404a is divided equally between all of the slashing stakeholders 404b (also detailed in FIG. 5B with regard to slashing module 518). Should the slashing bounty 404a exceed a predetermined amount, any additional accruals are directed to the DAO overseeing the information system base protocol. When a slashing event happens, all counters 410, 410′ are reset, and slashing pool balance drops to 0, which automatically deprioritizes the entry. Additionally, a slashing Boolean is activated to show that this entry was deemed to be poor quality by expert consensus.

FIG. 5A is a diagram of an exemplary incentive mechanisms of the system of the present invention. The goal of the question/entry incentive mechanism in the disclosed protocol is to surface the most popular questions among knowledge-seekers in a given knowledge area and the entry to that question that contains the most accurate and engaging answer from experts in that field. In this case, accuracy is defined as global expert consensus within a knowledge domain. This model has two main actors: the users 502, 504, and the experts 506, 508, 510. The users 502, 504, are incentivized to ask questions 512 that are most relevant to the broader user-base. The experts 508, 510 are incentivized to provide answers 514, 516 that are most aligned with the broader expert knowledge base.

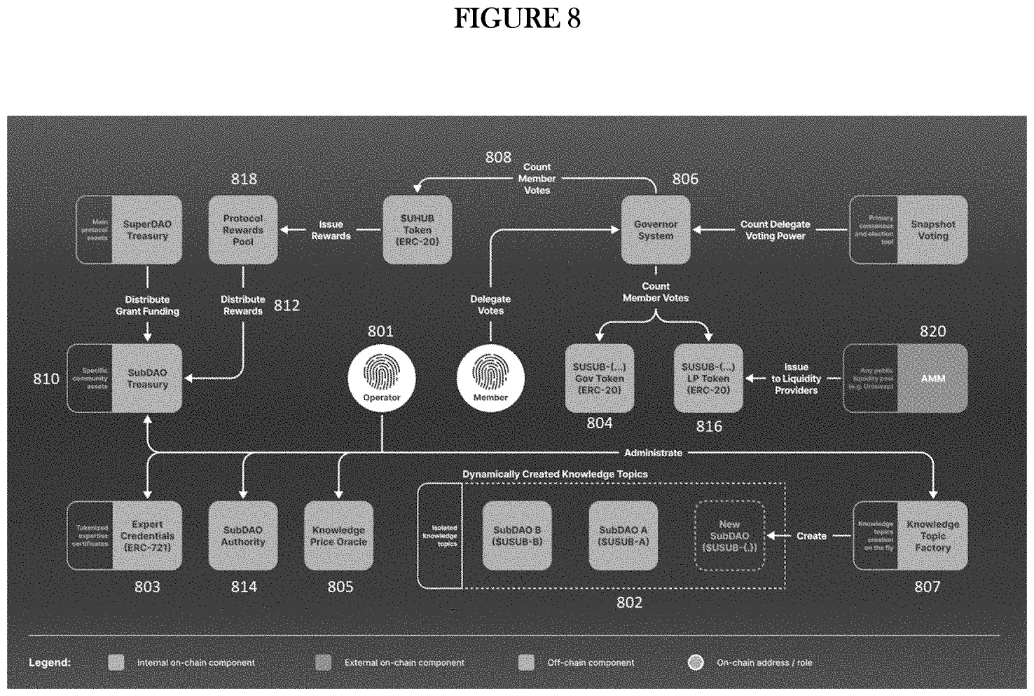

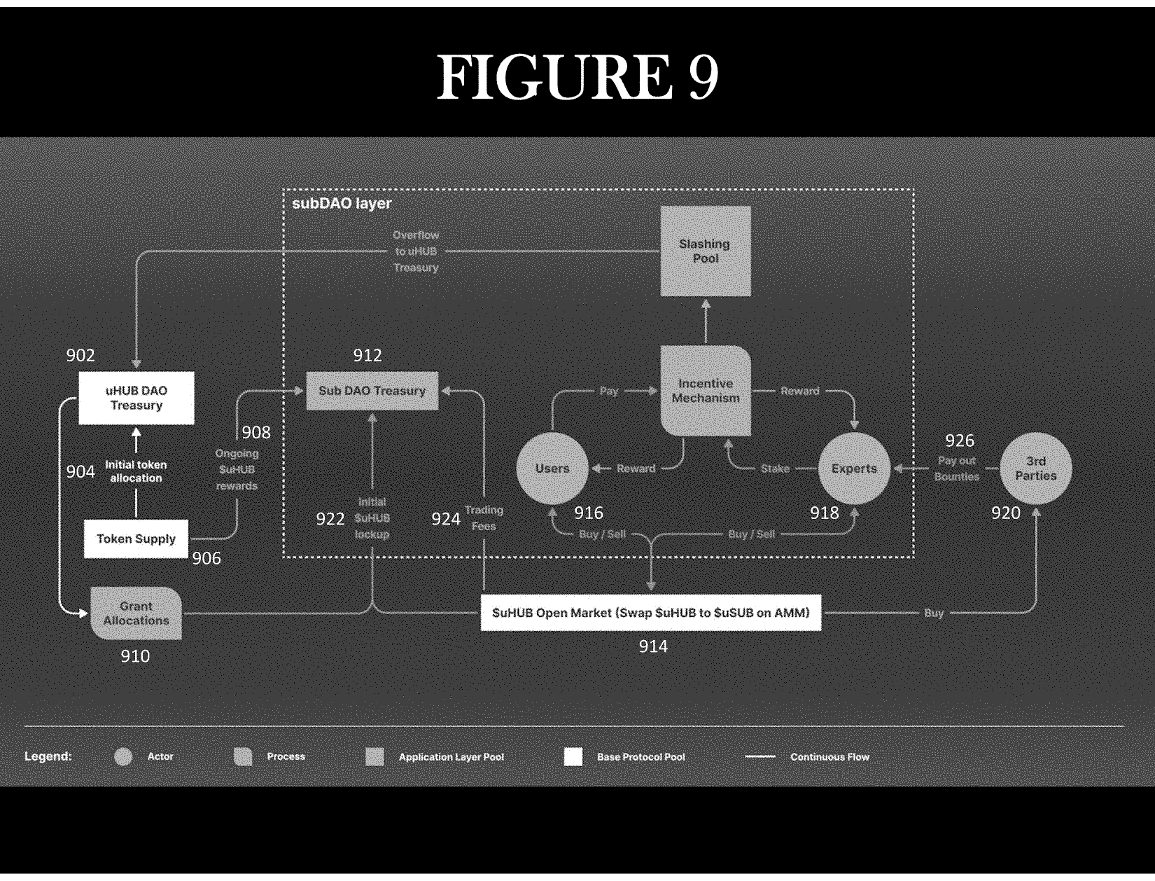

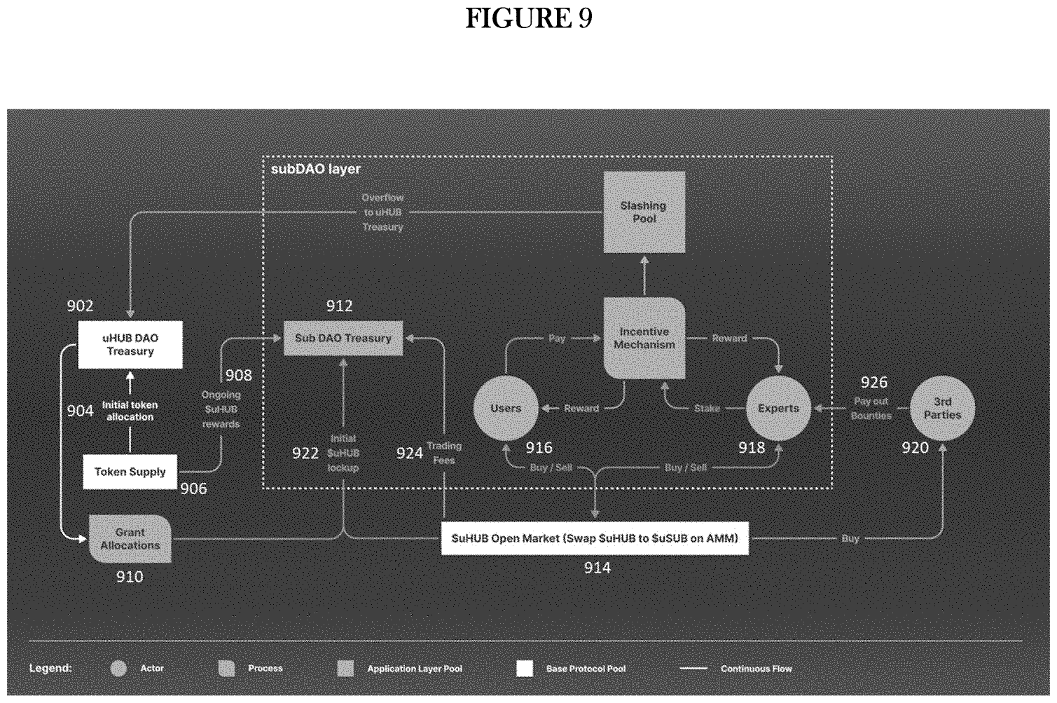

Once a user desires to access a specific query, several interactions take place, outlined in FIG. 5B. The user wallet via a front-end application 401, requests to access knowledge base 522 query address of interest. The knowledge base 522 calls the access module 526 to review the list of addresses authorized to access that query address in one embodiment this can be stored in the knowledge base as in shown in FIGS. 4 (403 and 404), while in another embodiment, the list of addresses can be stored in secondary database solely managed by the access module 526. The access module 526 returns to the knowledge base 522 a boolean value indicating whether the user address can or cannot access that query (discussed in detail in paragraph 0054). Assuming no prior access has been granted, the knowledge base 522 calls the knowledge price oracle 524 to determine the required fee (this is a parameter set by the subDAO responsible for maintaining the knowledge base). The price oracle queries a pre-specified set of markets/automated market makers/liquidity pools (shown as 820, 914 with regard to FIGS. 8 and 9 below) to determine the appropriate exchange rate in tokens and returns that value to the knowledge base 522. The knowledge base returns the needed value to the user wallet by initiating a smart contract with the user wallet that is signed by the payment transaction. In one embodiment this can involve the incentive module 532, the slashing pool 518, the access module 526, and the bounty module 536. In this contract the user fee is split between the query author address 402, the entry author address(es) 406, the slashing pool (and/or its associated DAO overflow address 538). The slashing pool will return the appropriate target for fund distribution described in paragraph 052. The access module receives no fees, but rather returns from the knowledge base the credentials needed to access that specific query. In some embodiments, each signed transaction can update that knowledge base, in other embodiments transactions are aggregated over time in the incentive module and the knowledge base is updated in bulk every epoch or every block within >1 transaction. At such a time, the funds are held in the incentive module are disbursed based on the parameters set forth in the knowledge base and any triggered bounty modules 406. This interaction between modules is mirrored when an expert contributes to the knowledge base 522, or a slasher votes to slash an entry in the knowledge base with various functions of each module being called to accomplish these tasks.

A question 512 can be asked in 2 formats: n:n or 1:n. The ratio represents the allowed users:experts for a given question 512. In this ratio n is an unlimited number. In the n:n case an unlimited number of users 502, 504 can access the answers 514, 516 and an unlimited number of experts 508, 510 submit an entry 514, 516. In the 1:n case, a single user 502, 504 can ask a question 512 and access the answers 514, 516 from an unlimited number of experts 508, 510 who choose to respond.

In the n:n case, a user 502 will ask a question for a small fee that gives them a claim to a small percentage of future access fees. This mechanism incentivizes users 502, 504 to ask questions that they believe will be most useful to the most people possible, even if the question 512 does not personally pertain to them. In a fully mature market, one could imagine individuals or businesses forming around researching and asking the most pertinent questions 512 to a given community which benefits all parties involved.

In the 1:n case, because only the asker of the question 512 can access the answers 514, 516, the higher cost to ask the question is not expected to have a financial return from future accessor fees. This user will pay the higher cost for the privacy of the answers returned. An example could be a company conducting a survey on devices that wants the answers from a panel of cardiologists but does not want their results accessed by other users. An alternative scenario is a user asking a question about a personal concern, seeking several opinions to gather a consensus from a community of experts. The fee offered for such questions is set by the user and is intended to work in a reverse-auction model, where higher fees are offered for more specific/complex questions

Experts 508, 510 will pay a small fee to post an entry to a question for a claim on a large percentage of the access fees paid by users 502, 504 in perpetuity. As more experts 508, 510 answer the question, the large percentage of the fees paid will be split among all answers, preferably weighted by the number of user endorsements (referred to as positive flags in previous sections/figures). This mechanism will naturally limit the number of answers that are economically viable to provide to a given question 512. The more answers provided, the less share the expert 508, 510 will receive overall. A very popular question will incentivize more answers 514, 516 from experts 508, 510, while an unpopular question will incentivize fewer answers as the rewards may not outweigh the initial fee.

A user 502, 504 will pay a very small fee to access all answers 514, 516 to a question 512 they are interested in. The incentive for this small payment will be to access a comprehensive and easily understandable entry rooted in expert consensus. A secondary interaction will be to flag the most useful entry to help prioritize it for future users and reward its expert author for their effort.

A motivation for the slashing mechanism of the present invention is to dis-incentivize answers that are exploitative or egregious enough that a sizable percentage of an expert community votes to have it removed. The mechanism to accomplish this consists of the slashing pool and slashing threshold 506.

A percentage of a given entry's endorsement-weighted revenue will be diverted into a slashing pool 518, 520 by the incentive module 532. The slashing pool contains a set of parameters that govern the maximal value of the pool (set by the subDAO), a list of addresses of slashing participants, and the address to direct excess funds to. When called by the incentive module, the slashing pool will return the appropriate destination for the slashing portion of the user's fee to be directed by the incentive module: the slashing pool fund address, or the DAO treasury address. If the transaction is with a slashing action, then the funds are directed to the slashing pool and the list of slashing participant addresses is modified. If there is a final slashing action that exceeds the threshold, the slashing contract returns a list of all slashing participant addresses for that knowledge base entry with the amount of the pool balance to be transferred to each one. The balance of the slashing pool 518, 520 grows proportionally with the popularity of a given entry 514, 516. This mechanism effectively burns tokens for good answers that are never slashed, which increases the value of a subDAO's economic unit. In order to slash an entry, experts 508, 510 are required to stake an amount into the slashing pool 518, 520 which becomes part of the bounty paid out to all stakers if the slashing threshold 506 of participating experts (set by the subDAO's operators via the subDAO's Knowledge Base 524) is reached. The total amount in a pool 518, 520 will be distributed equally among the experts who voted with their stakes 506. This distribution effectively redistributes value from all other token holders to experts acting as slashers via dilution. Experts 508, 510 are only incentivized to slash answers if they believe the slashing threshold for their community will eventually be reached for a given entry. If a slashing threshold is never reached, then the amount they staked for slashing is effectively burned and all other economy participants benefit. A slashing pool 518, 520 ceiling per entry will be determined by the system's governance (subDAO), which sets a cap for slashing pool value. This is set as a parameter in the knowledge base 522. This ceiling exists to prevent a slashing pool from reaching a value that incentivizes coordinated invalid slashing to redeem the rewards from an economically over-valued slashing pool. Any $uSUB tokens over the set ceiling will be claimable by the system base protocol treasury 538. This ensures that the system base protocol accrues value from successful management of information areas by their respective subDAOs and aligns the base protocol more strongly with the success of the subDAOs both from receiving $uSUB tokens over the slashing pool 518, 520 cap and growing their value and the information economy as a whole. The set of interactions described above will comprise the algorithms of a novel smart contract termed the slashing module 518.

The incentive module 532 manages fund escrow and disbursement. It is called by the knowledge base 522 and stores user funds 401 that it allocates based on the parameters of the knowledge base 522, slashing pool 518, and bounty module 536. The distribution of the contents of a transaction will occur based on queries of the query/entry combinations in the knowledge base. Address contents and tags from the knowledge base will determine the methods of fee divisions within the incentive module based on the access credentials and desired actions of the user (whether they be an expert user or a non-expert user). The output of the incentive module is the distribution of fee components (incentives) to the slashing pool, from the bounty pool, and to the addresses specified in the knowledge base.

The access module 526 will determine whether the user address interacting with the knowledge base has access to the area of the knowledge base. The module 526 is called by the knowledge base to assess the list of pre-existing addresses. It will also store algorithms for access to the encryption data 530 needed to access different number of entries and in doing so allow for a variety of pre-specified interactions (such as 1:1 and 1:n described above). The access module's permissions in the knowledge base will be to add to modify access credential metadata, return whether access is granted, and send credentials for accessing the out-of-chain storage media 528 that makes up the content of a single or group of query/entry combinations.

The bounty module 536 is designed to store funds from a commercial sponsor for the purpose of advertising within this blockchain. The sponsor can set any number of agreed-upon terms with each entry/author that will trigger financial release from the bounty based on user interactions with the knowledge system. It will be called by the incentive module to disburse the needed funds, and will determine the amount of funds based on user interactions within the knowledge base, i.e. flagging. Based on the interactions and the prespecified criteria, the bounty module 536 will then release funds to the incentive module 532 to be delivered to one or more expert addresses 506.

The knowledge price oracle 524 exists in many forms already, and unlike the knowledge base, incentive module, bounty module, it is not a novel concept. It is based off of pre-existing architecture for decentralized protocols and returns the exchange rate and token price of accessing an entry, staking fee for a new entry/query, and slashing price to the knowledge base.

Protocol-Based Management of User and Community Interactions

Protocol-based management of the incentive mechanisms described above occurs through the interaction of smart contract modules FIG. 5b. The core of every dedicated knowledge area is the Knowledge Base 522. This module encapsulates keeping the actual knowledge data references (such as links to questions and answers) along with the metadata that reflects the consensus around the quality and reputation of the knowledge illustrated in (FIG. 4).

The Knowledge Base 522 is the primary interface for the Users 502 and Experts 506 to:
Post queries
Post Entries
Positively and negatively flag entries
Get access to the existing knowledge
   This component's 522 primary role is to update the state of the system, rather than to calculate and distribute incentives. Fees charged for each interaction are in some embodiments pre-specified in the knowledge base, and other embodiments in the knowledge price oracle. Based on token exchange rate, the final token fee is and calculated within and returned by the knowledge price oracle 524 to the knowledge base 522 and is used to call the incentive module 532. The knowledge base 522 is also capable of modifying access to the access control module 526, which verifies access using off-chain storage 528 and a data encryption protocol 530.

The knowledge base 522 stores address data for processing by the incentive mechanism 532, as shown in FIG. 5B. The incentive mechanism 532 collects fees from the user address and bounty module and releases them to user 502, slasher 510 and/or expert 506 addresses, as well as the slashing pool 518 depending on the action being taken. In some embodiments, the bounty system 536 can be used to distribute revenue from the system to the subDAO treasury 538, 810. In other embodiments this may not be desired as it reduces the economic efficiency of experts within the ecosystem.

The way the system enforces the incentive policies is through the group of purpose-based incentive pools. The pool interfaces include Knowledge Staking 540—the pool that holds User 502 deposits required to ask a question, the Slashing Pool 518—the pool that Experts stake into to back the knowledge they generate, and the Bounty System 536—the pool where a third party can post a bounty, locking funds along with a specific task attached to it.

Incentive pools 518, 536, 540 keep funds and maintain the state of the unreleased incentives in the knowledge area until they are called by the Incentive Mechanism 532 to execute reward distribution or penalty slashing. Finally, the mechanism 532 calls the Incentive Pools 518, 536, 540 if necessary to execute the funds distribution or slashing, re-balancing the system to reflect the current value its Knowledge Base 522 provides.

Sitting on top of the Knowledge Base 522 and Incentive Pools 518, 536, 540, the Incentive Mechanism 532 is the engine that implements the tokenomics model and maintains equilibrium in the system, as shown in FIG. 5A. The mechanism runs every predetermined epoch, taking the latest metadata from the Knowledge Base 522 (such as reputation scores) and producing the incentive factors as the output.

In order to provide the protocol applications with a way to connect any peripheral data repository with the particular Knowledge Base 522, Access Control 522 component design lets the system maintain an on-chain user access list, mapping certain addresses to pieces of knowledge they have received authorized access to. One embodiment of this is using the access module to add a user's address to the paid access list 402b for all of the contents in that query, another can be a separate repository within the module of arrays of query identities and associated address lists. This module's smart contract in one embodiment calls the knowledge base contract to direct the user to the specific query or deny access to the application. In this manner, the Access control module facilitates any programmed (or curated) list of addresses to any manner of data storage repositories using credentials stored in the knowledge base to private database access or public storage data 528 encryption techniques 530.

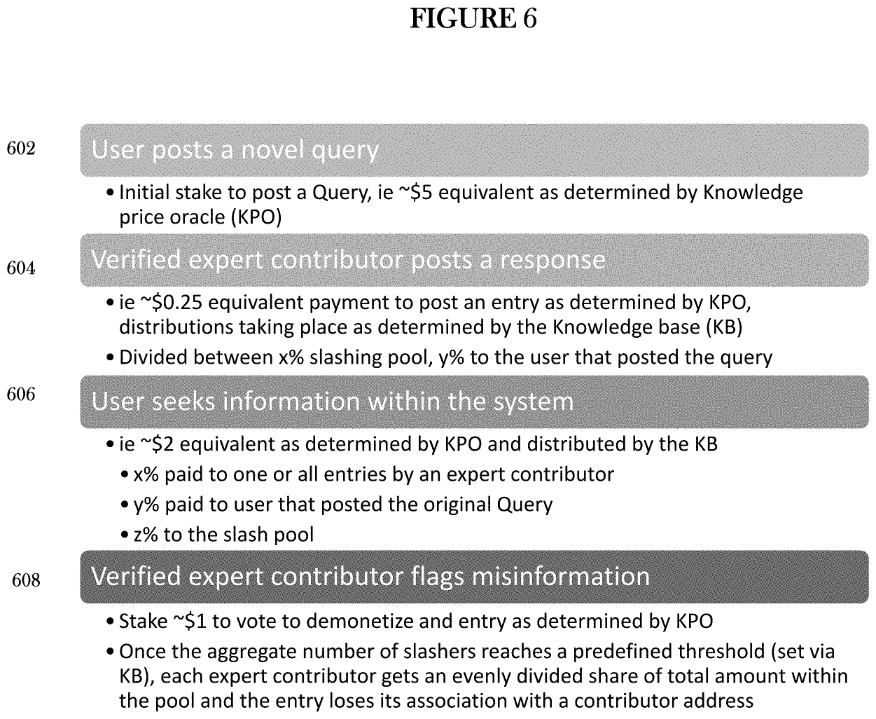

FIG. 6 is a summary diagram showing roles within the system and the economic transactions involved, displayed as an exemplary embodiment of the present invention. The reward framework depends on the type of user initiating an interaction within the system, and permissions that user type has to activate functionality within the system. If a general user posts a question into the system 602, then the system requires that user to pay a bounty, which is passed to the incentive module of the system, exemplarily in the cryptocurrency of native network (exhibited here as a USD equivalent). In certain embodiments, the fee is a fixed amount, though in other embodiments it may be variable. In response to such a user question, a verified expert contributor pays a fee to post an entry in response 604 that is partially or wholly reimbursed by funds left in the initial query bounty as computed by the incentive module. In certain embodiments, this fee is split between the author of the query 602 and the slashing bounty. When a user accesses the system of information on a posted question 606, that user must submit a fee into the system. The fees paid by the user accessing information are passed to the incentive module. The incentive module in one embodiment splits this fee between a single positively flagged entry and its associated expert contributor address, and in another embodiment where no entry has been positively flagged; evenly between all entries and their associated expert addresses. Additionally, the incentive module directs a portion of the user fee to the original author of the question, and a portion to the appropriate slashing bounty(ies). In one embodiment this is directed to the slashing bounty for the positively flagged entry, and in another embodiment split evenly between slashing bounties for all entries associated with the query (FIG. 4). In order to flag an entry for demonetization 608, a verified expert contributor must stake an amount in the slashing bounty of that specific entry (see FIG. 4). When the number of verified expert contributor staking within the slashing bounty reaches a pre-specified threshold, the slashing bounty is paid out evenly between all contributor addresses attached to the slashing bounty (FIG. 4). In some embodiments this will trigger a dissociation of funds generated by future interactions with the entry with its originating address. In other embodiments triggering the dissolution of the slashing bounty will negatively affect the reputation metric of its originating expert curator address. In other embodiments, the entry will have a demonetization/misinformation flare.

Administration of the Protocol

To effectively apply these knowledge curation incentives to the numerous communities of knowledge that form the basis of modern societies, the components of the incentive mechanism 532 are managed through a governance structure made of decentralized autonomous organizations (referred in this exemplar as a parent or super DAO and its subDAOs). Those organizations manage the balance of these incentives via Knowledge Price Oracles 524 that interact with the knowledge base 522, as well as the networks of experts 506 and users 502 that interact within a given knowledge space. This ensures that while the underpinning principles of the protocol remain static, their application to input/output modes, management of storage media, and adaptation to geographic, economic, and cultural variability is optimized.

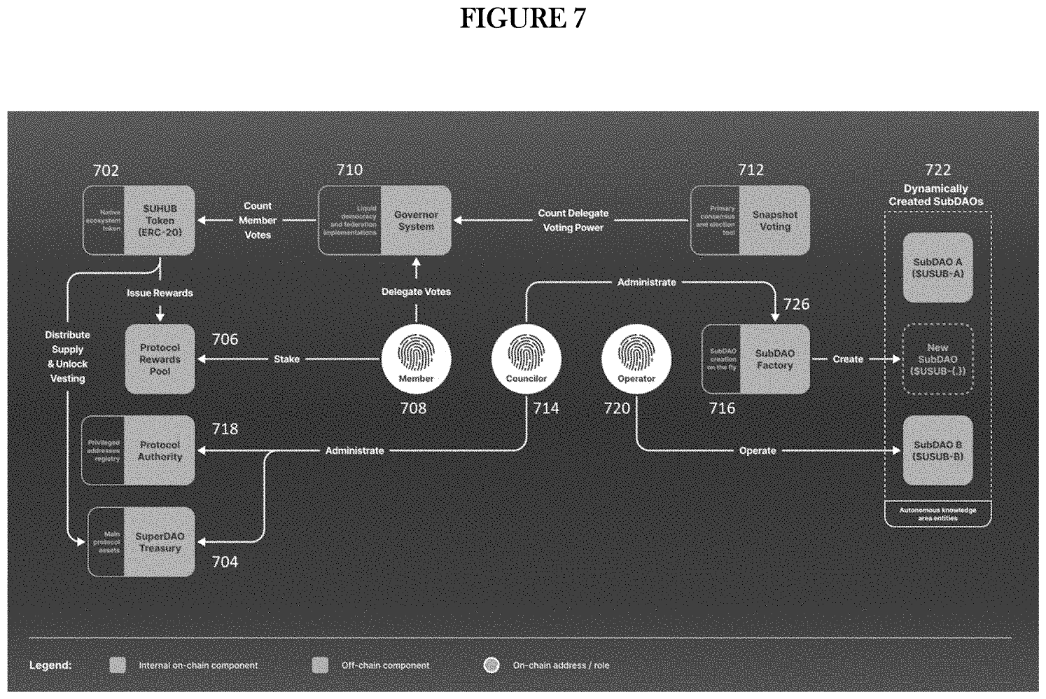

FIG. 7 outlines the organizational structures referred to above connected via a knowledge economy. A governance token 702 acts as the primary value accrual and transfer medium in the protocol, which in this exemplar can be referred to as the $uHUB token. Its design incorporates compliance with ERC-20 interface, as well as functionality that facilitates protocol-specific features such as vesting and time-lock. It allows for a multi-level protocol governance framework termed in this exemplar as the governor system 710 that makes it possible for the token holders to delegate their voting power to other addresses, as well as for the subDAO token holders to participate in top-level governance.

The DAO Treasury contract 704 is the recipient of the programmed protocol rewards and the depository for the protocol DAO funds managed through the governance process. The DAO treasury is used to financially support protocol maintenance and innovation, finance stewardship of the incentive module, seed the creation of new subDAOs, and act as a common repository for value for all participants in the protocol. The treasury is administrated by the DAO council 714 and the identities/roles of these councilors are managed by the Protocol Authority component 718. This serves as an on-chain broadcast beacon on which addresses are currently elected for the formal governance roles. Which are voted on in pre-specified intervals within the Governor system 710.

In order to facilitate the emission incentives and rewards mechanism across the ecosystem of subDAOs, the Protocol Rewards Pool component 706 acts as the receiver of the corresponding part of the $uHUB token supply. The pool smart-contract then distributes the rewards to the subDAOs 722 according to their performance measures against the current benchmark.

subDAO Architecture

As used herein, “subDAO” refers to a subordinate decentralized autonomous organization (DAO) protocol that helps expert communities through credentialing, off-chain storage subsidies, limited protocol parameter adjustments, and other community decisions related to the specific knowledge area around which the subDAO is organized. A subDAO is created by a smart contract that is deployed on a blockchain 726, and it has its own set of rules and governance structure. The parent DAO can exert a certain degree of control over the subDAO, such as setting certain parameters or making decisions about how the subDAO operates. SubDAOs are used to create a hierarchical structure within a decentralized organization, allowing for different levels of decision-making and control to allow the information system to serve knowledge communities of all sizes and backgrounds. In exchange for a portion of future economic activity the parent DAO provides an value foundation for a young community economy, where the demand for answers in exchange for a novel token that is backed by the larger information system economy represents a real inflow of capital that incentivizes early-stage participation from interested users and experts.

The design of the system of the present invention takes advantage of the modular software approach, enabling an unlimited number of autonomous organizations (referred to as subDAOs) to co-exist and evolve as parts of the ecosystem 802. The subDAO Factory component 807 is a primary tool for the DAO governors to establish new autonomously operated knowledge areas with modified economic incentives around them. Every such autonomous knowledge area 802 is isolated at the smart-contract implementation level sufficiently to have its own governance, treasury, tokenomics and user access modules configurable and controlled by the dedicated community formed around the knowledge area.

Every subDAO deployed on top of the main protocol is administrated by operators 801 (potentially organized into divisions/op units) who act as employees and executors of subDAO roles 803, 805, 807. These are operations are managed via a governance structure like the parent DAO, via its own governance token 804 that encapsulates the local governance process, as well as ensures that both governance and economic decision-making is sufficiently autonomous to allow independent operation. The token, however, remains a part of the larger ecosystem through a unified governance system 806, and emissions that are programmatically related to the main protocol. In addition to its own token, a subDAO operates an independent governor system 806 that resembles that of the main protocol. Both levels of governance are compatible with each other, giving the subDAO holders the right to also participate in the main protocol voting and delegation 808. The treasury managed by the subDAO holders 810 acts as the receiver of the main protocol reward distributions 812, as well as any grants or partner funds injections intended for the subDAO as a decentralized entity. The treasury is governed by the subDAO token holders and operated by the elected addresses via the governor system 806. Protocol Authority component 814 design at the subDAO level is identical to that of the main DAO.

One embodiment of subDAO operations is indirect stewardship over knowledge quality. On the blockchain, every subDAO keeps a map of protocol users and their access to the pieces of knowledge curated by the community. This map is meant to serve as the source of truth for the applications when managing access to the actual data storage. It is up to the subDAO to keep the knowledge access policy as tight or as liberal as they choose. This is accomplished via maintenance of Expert Credentials 803 that in some embodiments can be accomplished via issuance and storage of expert qualification certificates (Proof of Expertise) tokenized as ERC-721 standard based non-fungible tokens (NFT). This component acts as the on-chain source of truth for any other internal or external part of the system that relies on authorized access restricted to individuals with a certain level of expertise.

Another embodiment of subDAO operations involves managing their own knowledge market policies, slashing thresholds, and setting preferred pricing and fee structures via the Knowledge Price Oracle 805 component that the corresponding Knowledge Base relies on when charging users and experts for contributing to and consuming the knowledge via the interaction of the Knowledge Price Oracle 805 and incentive module.

An additional embodiment of subDAO functionality is the stewardship of a 3rd party bounty system FIG. 5b, 536 and FIG. 9, 926, developed in the application layer. This would include an automated ruleset (ex: “entry must include the word ‘cvs.com’,” “paid x $uSUB tokens per x user access,” etc.) and bounty pot of $uSUB tokens to be automatically claimed by experts that fulfill the bounty requirements. This system will be self-regulated by the consensus mechanism, and any egregious advertisements will be slashed if the community thinks they are shilling or an entry is swayed by its bounty.

Economics and Treasury Management

In one embodiment, as shown in FIG. 9, the parent protocol treasury 902 can receive an initial token allocation 904 from the token supply 906. Outside of the initial token allocation 904, the token supply 906 is a pre-programmed protocol-based token creation designed to constrict emissions in a programmatic, time-based manner, that in some embodiments can follow an equation that factors in network productivity such as the one outlined below.

$$y = x * {z}^{a ( \frac{1}{p} )} \\ y = emission at time a \\ z = constant multiplier \\ x = starting sum for an emmision \\ p = accrual of uSUB token value into parent treasury per unit time$$

A token emission 908 is further programmed to be divided between subDAO treasury addresses 912 based on $uSub contributions over a unit time to the uHUB treasury. The parent DAO treasury 902 grants can also give allocation 910 to a subDAO treasury 912. Another source of subDAO treasury revenue comes from liquidity provider (LP) fee generation generated from decentralized exchanges/automated market makers 914. These facilities 914 enable trading on the open market where any participants 916, 918, 920, 902, 912 can acquire or divest tokens in line with their interests in system engagement. For example, third parties 920 can pay out bounties 926 to experts 918 in exchange to responses to queries.

The subDAO treasury's 912 role is quite different, and as such, the subDAO treasury 912 will be controlled by the $uSUB holders (shown as 804, 816 in FIG. 8) of the respective knowledge subDAO. $uSUB is the medium of exchange upon which the incentive mechanism operates 818. In addition, it operates as the funding stream for operation of the subDAO, facilitating continuous decentralized data storage, paying DAO members for working for the subDAO and financing the development of application-layer specific functionality among other any number of roles. As touched on briefly in previous sections, in some embodiments, $uSUB creation is programmatically linked to $uHub lockup, thus the value of $uSUB will always be at least the value of the staked $uHUB, whereas accruals in value to $uSUB from usage demand will positively influence the $uHUB economy and in this manner benefit all stakeholders (including other subDAOs) across the ecosystem.

An actor(s) (e.g. an Ethereum address) can lock-up 922 any amount of $uHUB tokens to start a knowledge field. Upon locking these $uHUB tokens, the subDAO factory smart contract (FIG. 7, 726) will spin up a subDAO treasury with a 1:n priced Uniswap LP of $uHUB/$uSUB (FIG. 8, 816) to be locked for the duration of the subDAO's existence. This actor will also receive 2-year vested $uSUB tokens to their personal wallet, equal to half the amount of $uHUB they locked to start the subDAO. This effectively gives the creator 25% of the initial subDAO token supply that vests after 2-years. This gives the creator of a subDAO potential upside in the future and strongly aligns them with the success of the subDAO they created.

As users trade $uHUB and $uSUB, fees will be generated by the LP position which can be claimed by the subDAO into their treasury. The $uHUB locking requirement prevents spam subDAO creation and will increase the value of the $uHUB tokens as more subDAOs are created by the protocol. A subDAO may decide to lock more than the minimum amount if they are particularly optimistic that they will cultivate a thriving community. If a subDAO wishes, at any time, they can vote to mint new $uSUB tokens by trading $uHUB tokens to the base protocol DAO, at the initial $uSUB:$uHUB mint ratio. This would be useful in the long-term case that a large portion of $uSUB is locked in slashing pools and there isn't enough liquidity in the community's ecosystem to operate properly. This can be conceptualized as the subDAO voting to redeem value to the treasury from effectively burned tokens.

One important consideration from the architecture perspective is that a transition from the initial representative democracy framework to a more collusion-resistant liquid democracy framework has been factored in the design of the protocol architecture. The clear separation of the subDAO infrastructure modules—along with the isolated incentive mechanisms and equity represented by a dedicated governance token—makes the transition to the fully automated on-chain governance proposal execution mechanisms more effective.

Governance Overview

$uHUB 1004

The $uHUB token is the token of the base protocol and represents:
Voting on constitutional changes
Voting on grant allocations
Voting on base protocol treasury management
Locking to start a subDAO
A share of uHUB treasury reserves

$uSUB 1006, 1008

The $uSUB token is a generalized name for a subDAOs native token (ex: $uMED, $uBREAD, etc). This token can be used for:
The unit of exchange within the information ecosystem of the subDAO, used in computations within the incentive module and associated rewards to users and experts
All of the same functions as the $uHUB token except “Locking to start a subDAO”
Voting on application-level parameter changes
Voting on subDAO treasury management
Voting on expert credentialing process

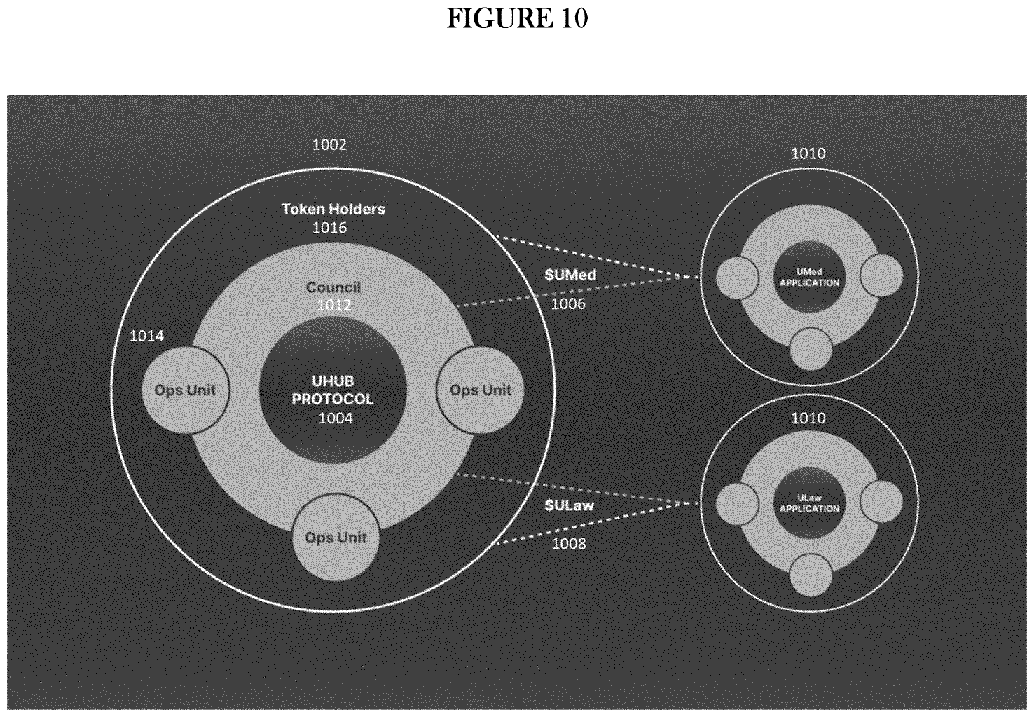

UHub's governance consists of 3 distinguishable units (FIG. 10): DAO Participants 1016, Council 1012, and Operational Units 1014. The combination of these units into a governing body will be the framework for a tiered system of governance from the original DAO to any number of subDAOs 1010. Each governance group is held together by a common set of rules, the Constitution. The original constitution can be crafted by the founders of the information system, but to ensure adaptability, subsequent proposals and amendments can be updated with proposals from DAO participants 1016 with a 1:1 reference to the current version. The Council 1012, as the oversight body, provides direction to the DAO and is equipped with predefined governance rights, that can include but are not limited to staffing operational units or triggering dispute resolution. Members 1016 chosen to be in the Council will be required to disclose their identities publicly and terms would be time-limited and extended by re-election, and once elected, council members 1012 can be issued a badge (e.g. ERC-721 standard-based NFT) as on-chain proof of this status and the rights associated with it. Operational units 1014 are established by the Council and can include but are not limited to marketing, development & account Management, human resources. Members of operational units are appointed/recruited by the council in much the same way as employees are hired in any organization, these positions would be considered key to DAO daily functions, and as such do not have a mandate for term limits. Contributors to the organization can be either organization members or non-affiliated contributors. Any contributions to the function of the organization or protocol will be attached to on-chain reputation and in some embodiments can be recognized through NFT badges such as those issues through the proof of attendance protocol (POAP).

DAO participants 1016 include all token holders of the native UHub token 1004 as well as the subDAO tokens 1006. They are the organizational corrective and have voting rights on fundamental decisions, such as changes of the Constitution and leadership changes similar to the role of shareholders in a traditional corporate economy. Each token holder starts out with the logic “one token—one vote” to influence outcomes of elections following a “relative quorum” model. Here, a proposal is accepted if 51% of the participating voters vote in favor of it. To empower the most committed and experienced DAO participants, improving the overall governance quality, “Reputation” can be factored into the allocation of voting power. Detailed thresholds for allocation of voting power in relation to on-chain reputation metrics can be set in the constitution, with considerations in for factors that include but are not limited to scope of contribution, length of engagement, lock-up amounts and timing (ie: modifications to vesting scheduled), etc. Lastly, effective governance can be encouraged through options for delegation of voting power (as facilitated by the governor system 806) to dedicated governance experts (individual or group entities) acting as community delegates.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. An information system comprised of one or more servers that:
receive one or more information queries, wherein the information queries are associated with a first set of smart contracts that are comprised of economic incentives, access credentials, and governance roles;
receives one or more responses to the information queries, wherein the responses are stored as a second set of smart contracts;
requests validation of the responses from interactions with other smart contracts in the blockchain, wherein the validation request is processed using knowledge-community managed smart contracts and economically aligned contracts; and
analyzes user queries for relevance and ranks responses to the information queries using a neural network algorithm uniquely generated from iterative user-system interactions; and
outputs the responses to the information queries and distributes economic incentives in line with community-specified smart contract requirements.

2. The system of claim 1, wherein the information queries are transmitted to the server with a fee.

3. The system of claim 1, wherein the knowledge-community managed smart contracts utilize a knowledge price oracle module.

4. The system of claim 1, wherein the economically aligned contracts are calculated by an incentive module.

5. The system of claim 1, wherein the information queries are affiliated with a transmitting user's identity and receive future royalties derived from paid access to said queries by other users.

6. The system of claim 1, wherein expert contributors stake fees to post responses to the information queries.

7. The system of claim 1, wherein the information queries are affiliated with a transmitting expert's identity and receives future royalties derived from paid access to said information query by other users and bounties from third parties.

8. The system of claim 1, wherein the information queries are affiliated with a transmitting expert's identity is subject to removal from the incentive economy via group consensus by other experts within the information system.

9. The system of claim 1, wherein the information queries are affiliated with a transmitting expert's identity has a pool of economic value that receives royalties derived from paid access to that entry by other users, wherein the royalties are distributed to experts who deem that entry to be inaccurate by group consensus.

10. The system of claim 1, wherein the information queries are affiliated with a transmitting expert's identity has a pool of economic value that receives royalties derived from paid access to said information queries by users, wherein the system transfers royalties to a governing organization once a sufficient economic value in the pool has been reached.

11. The system of claim 1, further comprising a knowledge base module that stores actual knowledge data references.

12. A method of managing information comprising:
receiving one or more information queries, wherein the information queries are associated with a first set of smart contracts that are comprised of economic incentives, access credentials, and governance roles;
receiving one or more responses to the information queries, wherein the responses are stored as a second set of smart contracts;
requesting validation of the responses from interactions with other smart contracts in the blockchain, wherein the validation request is processed using knowledge-community managed smart contracts and economically aligned contracts; and
analyzing user queries for relevance and ranks responses to the information queries using a neural network algorithm uniquely generated from iterative user-system interactions; and
outputting the responses to the information queries and distributes economic incentives in line with community-specified smart contract requirements.

13. The system of claim 1, wherein the information queries are transmitted to the server with a fee.

14. The system of claim 1, wherein the knowledge-community managed smart contracts utilize a knowledge price oracle module.

15. The system of claim 1, wherein the economically aligned contracts are calculated by an incentive module.

16. The system of claim 1, wherein the information queries are affiliated with a transmitting user's identity and receive future royalties derived from paid access to said queries by other users.

17. The system of claim 1, wherein expert contributors stake fees to post responses to the information queries.

18. The system of claim 1, wherein the information queries are affiliated with a transmitting expert's identity and receives future royalties derived from paid access to said information query by other users and bounties from third parties.

19. The system of claim 1, wherein the information queries are affiliated with a transmitting expert's identity is subject to removal from the incentive economy via group consensus by other experts within the information system.

20. The system of claim 1, wherein the information queries are affiliated with a transmitting expert's identity has a pool of economic value that receives royalties derived from paid access to that entry by other users, wherein the royalties are distributed to experts who deem that entry to be inaccurate by group consensus.

21. The system of claim 1, wherein the information queries are affiliated with a transmitting expert's identity has a pool of economic value that receives royalties derived from paid access to said information queries by users, wherein the system transfers royalties to a governing organization once a sufficient economic value in the pool has been reached.

22. The system of claim 1, wherein a knowledge base module stores actual knowledge data references.
